(19) United States
(12) Patent Application Publication
(10) Pub. No.: US 2008/0177220 A1
(43) Pub. Date: Jul. 24, 2008

(54) Ultrasound-Mediated Transcleral Drug Delivery

(75) Inventors: Daniel Lindgren, Kansas City, MO (US); Ashim K. Mitra, Overland Park, KS (US)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: THE CURATORS OF THE UNIVERSITY OF MISSOURI, Columbia, MO (US)

(21) Appl. No.: 11/620,990

(22) Filed: Jan. 8, 2007

Related U.S. Application Data
(60) Provisional application No. 60/756,897, filed on Jan. 6, 2006.

(51) Int. Cl. A61F 9/00; A61M 31/00

(52) U.S. Cl. 604/22

(57) ABSTRACT

The present invention relates to processes, systems, and apparatuses for transcieral delivery of pharmaceutical formulations to the eye using ultrasound. In one embodiment, a transducer is placed in contact with a coupling media contained in a coupling well in contact with the sclera. When the transducer is placed at a desired standoff distance, ultrasonic waves are emitted to increase tissue porosity and transport a pharmaceutical formulation through the scleral tissue and into the eye. In another embodiment, a function generator is coupled to an amplifier, a matching network, and a transducer configured to maximize the cavitation effect of ultrasonic waves for drug delivery across a sclera.

### CROSS-REFERENCE TO RELATED APPLICATIONS

This application claims the benefit of U.S. Provisional Application Ser. No. 60/756,897 filed Jan. 6, 2006 and entitled “Ultrasound-mediated Transcleral Drug Delivery,” which is hereby incorporated herein by reference.

### STATEMENT REGARDING FEDERALLY SPONSORED RESEARCH OR DEVELOPMENT

Not applicable.

### BACKGROUND OF THE INVENTION

Treatment of various illnesses and ocular disorders often requires targeted delivery of pharmaceutical agents to the back of the eye. Age-Related Macular Degeneration, the number one cause of blindness, Diabetes Mellitus, and Herpes Cytomegalovirus are examples of illnesses and disorders for which targeted drug delivery to the eye is desired. Currently, unreasonably invasive routes of administration are used. Systemic drug delivery can be an option for certain pharmaceutical agents, but can result in undesired side effects in non-targeted tissue and low bioavailabilty in targeted tissue. Tissues in the back of the eye, including the retina, the retina pigment epithelia, the choroid, and the macula are primarily responsible for supporting the rod and cone activities associated with translating light signals into vision. Many ocular disorders affect these tissues and require invasive treatment. These tissues must be targeted for delivery of pharmaceutical formulations to treat many ocular disorders.

Currently, painful intravitreal injections are used to deliver drugs to the vitreous of the eye. After injection, the drugs must then diffuse through ocular tissue to reach targeted locations. These injections are painful, resulting in low patient compliance, and inefficient, requiring imprecise diffusion through ocular tissue. Fallout rates for patients in clinical testing have made patient compliance a significant practical problem associated with delivery of various ocular treatments. A less painful, more precisely targeted method of delivering drugs to the eye is needed to overcome these obstacles.

Ultrasound has been investigated as a means of transporting pharmaceutical agents across various physiological barriers and through various tissues. For example, ultrasound can be used to transdermally deliver drugs through the skin, as disclosed in U.S. Pat. No. 5,656,016 to Ogden. Ultrasonic waves have the ability to alter tissue porosity and increase tissue permeability allowing pharmaceutical formulations to diffuse across tissue barriers at much faster rates than topical application alone. Permeability, flux, and concentration have been significantly enhanced for several classes of pharmaceutical agents by using concurrent application of ultrasound during administration of the agents. One application of transdermal ultrasound drug delivery has been to deliver localized anesthetic agents to decrease sensation prior to injections.

The effect of ultrasound can vary greatly for various pharmaceutical formulations and biological barriers. When exposed to ultrasound, hydrophilic and hydrophobic drugs diffuse differently across various biological barriers depending on a number of factors, including the specific tissue barriers present as well as other transport phenomena that are occurring simultaneously.

Ocular applications have received some limited attention. For example, the effect of ultrasound on corneal tissue permeability has been investigated in rabbit models. It has been demonstrated that ultrasound can increase the porosity of corneal tissue to enhance transport rates across corneal tissue. Though this effect has been demonstrated, diffusion of pharmaceutical agents through the cornea to the inner eye requires transport across numerous layers of tissue and is poorly suited for delivery of agents to posterior regions of the eye. For transport to the inner eye via the cornea to occur, several anterior tissue barriers must be overcome. First, the epithelial layer of the cornea must be crossed. The epithelial layer inhibits transport of most hydrophilic drugs. Next, the stroma of the cornea must be crossed. The stroma inhibits transport of hydrophobic drugs. After the agent crosses the stroma, it must clear the endothelial layer, which is located on the interior of the cornea. After successful transport across the cornea, the agent is delivered into the aqueous humor between the cornea and lens. Then, the pharmaceutical agent must pass either through the ciliary body or potentially around the lens of the eye, so it can enter the vitreous. After the agent diffuses through the vitreous, it must then cross the outer layer of the retina pigment epithelium. Only at this point, is the pharmaceutical agent finally at the pathogenic point of interest, which is usually the macula, where most ocular pathogenic disorders originate.

It has been demonstrated that ultrasound can be used to increase the rate of transport of various pharmaceutical agents through corneal tissue, however a more effective method of delivering pharmaceutical agents to the posterior segment of the eye and the macula, in particular, is needed. Different types of tissue and physiological barriers must be overcome to deliver pharmaceutical agents effectively to these regions of the eye. A method capable of overcoming these barriers is needed.

### SUMMARY OF THE INVENTION

In embodiments of the present invention, methods for performing ultrasound-mediated transcleral drug delivery are provided. An ultrasound device is placed in contact with a coupling media containing a pharmaceutical formulation within a coupling well. The device is positioned at a desired standoff distance from the sclera of an eye. Ultrasonic waves are generated to increase tissue porosity and transport the pharmaceutical formulation through the sclera and into the eye. In embodiments, a pre-configured cartridge can be used to position the device at a pre-determined standoff distance.

In other embodiments, ultrasonic transcleral drug delivery systems and apparatuses are provided. In one embodiment the system comprises a function generator is coupled to an amplifier, a matching network, and a transducer. The system is optionally coupled to a visual display or oscilloscope. The system generates a desired electrical function signal, amplifies the signal, matches it, and converts it to ultrasonic radiation. The transducer and function are configurable for the desired drug-delivery application. In embodiments, the system can operate in a pulsed, continuous, or combination pulsed-continuous mode and at a plurality of frequencies. In embodiments, the transducer has a tip with a concave surface that closely corresponds with the curvature of an eye at its sclera.

### BRIEF DESCRIPTION OF THE SEVERAL VIEWS OF THE DRAWINGS

The present invention is described in detail below with reference to the attached drawing figures, wherein:

FIG. 1 is a diagram illustrating a method for transcleral drug delivery using an ultrasound device;

FIG. 2 is a diagram of an exemplary ultrasound transcleral drug delivery system;

FIG. 3 is a graph displaying the permeability-enhancing effect of applying ultrasound to a simulated tissue membrane; and

FIG. 4 is a graph displaying the effect of ultrasound standoff distance on the permeability of retina, choroid, and sclera tissue.

### DETAILED DESCRIPTION OF THE INVENTION

Embodiments of the present invention provide processes and apparatuses for delivering pharmaceutical agents across the sclera of an eye using ultrasound. In one embodiment, an ultrasonic device, such as a transducer, is placed in contact with a coupling media contained in a well that is in contact with an eye. The coupling media can contain various forms of pharmaceutical formulations to be delivered to various parts of the eye. The ultrasonic device emits ultrasonic waves, which increase tissue permeability and flux, to substantially increase the rate of delivery of the pharmaceutical formulation. This method is advantageous over topical application, intravitreal injection, and transcomeal delivery, which all have numerous setbacks that are overcome by the present invention.

Ultrasound-mediated transcleral drug delivery (UMTDD) can be used to deliver various pharmaceutical agents to targeted ocular tissue. UMTDD refers to the process of using an ultrasound source to enhance delivery of drugs or pharmaceutical agents across the sclera portion of an eye. The terms “agents,” “drugs,” and “formulations” will be used interchangeably. Fewer tissue and physiological barriers and different types of tissue and physiological barriers must be overcome by this transcleral ultrasound delivery method than by using other ocular delivery methods. The terms tissue barrier and physiological barrier will be used to describe various biological barriers which can tend to inhibit transport of types of matter to targeted locations. These barriers include both physical tissue layers as well as simultaneously occurring transport phenomena that tend to inhibit or counteract the desired drug delivery processes.

A transcleral transport pathway involves diffusion of the pharmaceutical formulation first across the conjuctiva, an external tissue layer where tear clearance presents a physiological barrier to drug delivery. Topical application of pharmaceutical formulations are often quickly cleared by tear action. However, application of ultrasound can overcome this tear clearance issue. After crossing the conjuctiva, the agent must cross the sclera, which is a hydrophilic layer. After crossing the sclera, the agent crosses the choroid, followed by the blood-retina-barrier, and then diffuses into the vitreous cavity, where it can reach the retina. This transcleral route involves crossing different, yet fewer biological barriers to achieve delivery of the pharmaceutical agent to the interior of the eye.

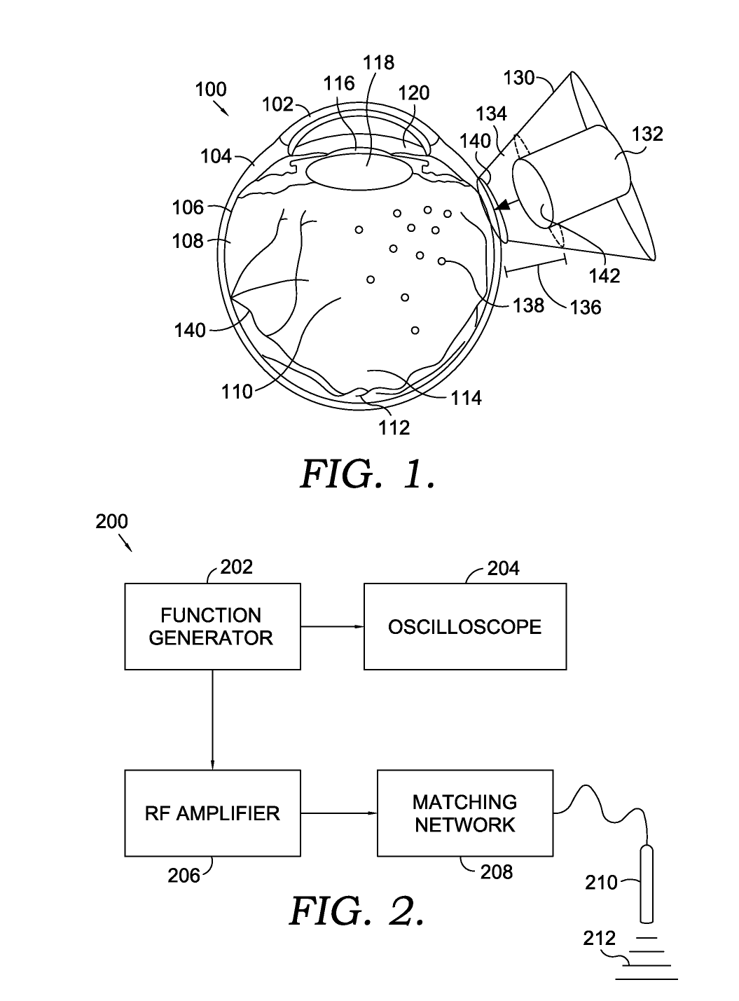

FIG. 1 displays an exemplary method set-up 100 for delivery of pharmaceutical formulations across the sclera of an eye using an ultrasound drug delivery apparatus for performing embodiments of the present invention. A coupling well 130 is placed in contact with the sclera 104 of the eye and filled with a volume of coupling media 134. The coupling well 130 can take any shape that accommodates holding a volume of coupling media. The coupling well 130 can also be a pre-configured cartridge, as described below. The exemplary well 130 has an open coupling end 140 with a desired cross-sectional exposure area to allow the coupling media to be in contact with the eye. In this embodiment, the well 130 has a uniformly, gradually increasing diameter farther from the coupling open end, which creates a conical shape. It should be noted that the coupling well 130 need not be conical in shape and is merely exemplary in nature.

The coupling media 134 provides a medium to transmit the ultrasound waves to the sclera, which is also in contact with the media. An optimum coupling media translates as much energy from the ultrasound waves as possible. The coupling media 134 can be, by way of example and not limitation, an aqueous media or a lipophilic media. Various ultrasound coupling agents capable of serving as coupling media are well known in the art. In embodiments of the present invention, the coupling media contains the pharmaceutical formulation to be delivered to the eye through the sclera. For example, the agent can be in solution in the coupling media or can be delivered in microcarriers, such as by being bound to the surface of microcarriers, contained in pores of the microcarriers, or encapsulated by the microcarriers. A method such as High Intensity Focused Ultrasound, which is known in the art, can be used to alter the agent once it has diffused through the tissue. This is another means of drug delivery. The appropriate coupling media can differ depending on the specific application. For example, the coupling media used can differ depending on the particular desired pharmaceutical formulation being delivered. The coupling media can also be optimized for stability and for maximum transmission of ultrasound to the sclera. And, the coupling media can be gas saturated to improve the cavitation activity at the interface of the sclera and coupling media. A sufficient volume of coupling media 134 is placed into the coupling well 130 to allow for a desired standoff distance 136 as well as to facilitate transport of the particular form of pharmaceutical agent (e.g., additional coupling media may be required if a particular pharmaceutical formulation is to be delivered in solution and the agent happens to have a lower solubility).

As discussed above, the coupling well 130 can also be a cartridge. The cartridge can position an ultrasound transducer 132 to have a pre-determined standoff distance. This allows cartridges of varying standoff distances to be used for different applications. For example, one standoff distance can be used for one particular pharmaceutical formulation while another standoff distance can be used for another particular pharmaceutical formulation. Formulation and application specific cartridges can be used. In embodiments, pre-configured cartridges can be used to provide pre-determined standoff distances, pre-determined amounts of surface area contact with the sclera, and pre-determined coupling well volumes to allow for sufficient volumes of media and pharmaceutical formulation to be used. By way of example and not limitation, pre-configured cartridges that position the transducer tip at distances of 0.50, 1.00, and 1.50 centimeters, respectively, can be used. These pre-determined settings allow for formulation and application specific cartridges to be used to optimize and standardize delivery of particular agents under for particular circumstances. Additionally, the cartridges can include a transducer-specific connector, such that the cartridge is “keyed” to the transducer. This connector ensures that only the appropriate transducer for that application-specific cartridge can be used. In this embodiment, the cartridge is shaped to provide the appropriate surface area for drug delivery and ensures that the area of exposed sclera is optimized to control consistent dosing concentration over time. In other embodiments, the cartridge can be adjustable to provide multiple settings for standoff distances. For example, the cartridge can be configured to provide for standard standoff distances of 0.50, 0.75, 1.00, 1.25, and 1.50 centimeters. A single, adjustable cartridge allows for multiple pre-set standoff distances to be used without the need for individual cartridges at each distance. Via combination of standoff distance, transducer-specific connector, surface area, shape, and exposure time, these embodiments allow controlled delivery of pharmaceutical agents in a repeatable method.

An ultrasound transducer 132, which is part of an ultrasound system 200 as discussed below with reference to FIG. 2, is placed in contact with the coupling media 134 inside the coupling well 130. The transducer 132 is positioned so as to achieve a desired standoff distance 136. In an embodiment in which the coupling well 130 is a cartridge having pre-determined settings, the standoff distance 136 is a pre-determined standoff distance. In this embodiment, the cartridge enables the contact between the coupling media 134 and the transducer 132, as well as the contact between the coupling media 134 and the sclera 104. The transducer 132 converts electrical energy wave functions into ultrasound waves and emits the waves through the coupling media 134. The ultrasound waves temporarily alter the porosity of the ocular tissue to substantially enhance transport of the pharmaceutical formulation 138 into the eye.

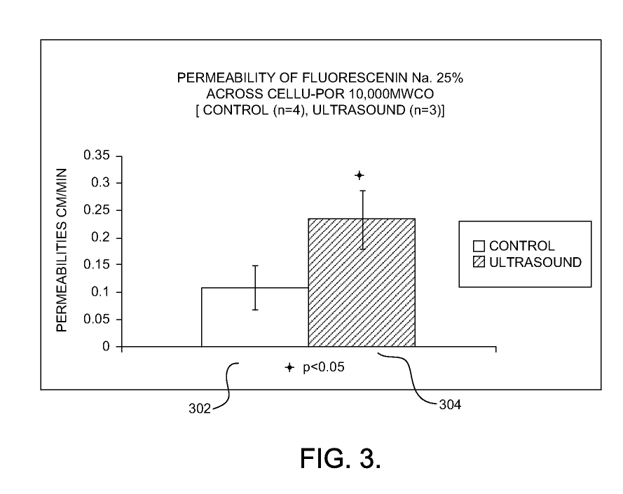

The impact of ultrasonic waves on diffusion of pharmaceutical formulations is shown in FIG. 3, which displays experimental results for diffusion of a Sodium Fluorescein formulation across a Cellu-Por synthetic membrane that simulates ocular tissue. The control results 302 display the effect of allowing the formulation to diffuse naturally, while the ultrasound results 304 display the effect of applying ultrasound concurrently during application of the formulation. As shown in FIG. 3, substantially higher permeabilities are achieved when transporting the agent using ultrasonic waves.

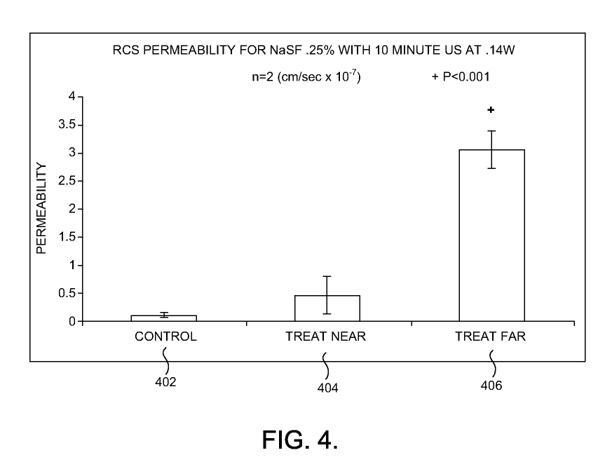

By using a standoff distance 136, drug transport can be optimized. A standoff distance is desired to optimize the cavitation effects in the Fraunhofer zone of the ultrasonic energy field. The standoff distance 136, or distance of the transducer tip from the surface of the sclera, can impact the permeability, or the rate of drug delivery, through the sclera., as shown in FIG. 4, which displays experimental results for diffusion of a Sodium Fluorescein formulation through retina, choroid, and sclera (RCS) tissue from New Zealand albino rabbits in a Franz diffusion cell. The control results 402 represent normal diffusion action of the formulation in the absence of ultrasound. The treat near results 404 represent diffusion of the formulation achieved using a 0.50±0.01 cm standoff distance. The treat far results 406 represent diffusion of the formulation achieved using a 1.00±0.01 cm standoff distance. As the results show, substantially higher permeability (approximately 30 times higher) was achieved using the greater standoff distance. The optimum standoff distance can vary depending on a number of factors, such as, for example, the coupling media, the transducer configuration, the targeted tissue, and the pharmaceutical formulation being administered. The optimum standoff distance for transcleral drug delivery differs from drug delivery attempted through the cornea due to the numerous factors discussed above, including inherent tissue differences and transport phenomena occurring in the blood-retina barrier.

After the coupling well 130, coupling media 134, and transducer 132 are in place at the desired standoff distance 136, sonication is applied for a desired exposure time. The desired exposure time varies based upon the particular drug, the desired concentration to be achieved, and the tissue. In general, as longer exposure time is used, higher concentrations of the transported drug are achieved. Some embodiments of the present invention use an exposure time of 10 minutes. Other embodiments use an exposure time of between 20 seconds and 10 minutes. This time is advantageous over intravitreal injection, because, the combined preparation time and injection time for intravitreal injection is often well in excess of 10 minutes. In addition, the substantially lower pain levels involved with ultrasound delivery relative to intravitreal injection make patient compliance substantially higher regardless of any lengthy exposure time required.

In embodiments of the present invention, an ultrasound frequency of 750 KHz is used, while other embodiments of the present invention use an ultrasound frequency of 1 MHz. Still yet other embodiments use a broad range of potential frequencies, but an upper limit exists where tissue begins to be irreversibly altered and where thermal effects are unacceptably high. A one degree Celsius thermal effect is a desired upper limit.

The exemplary ultrasonic transcleral drug delivery system 200, shown in FIG. 2, can be used to perform an ultrasound-mediated transcleral drug delivery process. The system 200 comprises a function generator 202, an oscilloscope 204 or other function display device, an amplifier 206, a matching network 208, and a transducer 210. The function generator 202 is used to generate electrical energy at certain frequencies and certain levels according to a designated algorithm. The function algorithm can be optimized based on the particular application. For example, certain pharmaceutical formulations and certain tissues may be more responsive to particular functions. The frequency range of the exemplary function generator 202 is 1 KHz to 21 MHz and its amplitude range is 1 mV to 10V p-p. The oscilloscope 204 can be any device capable of generating a visual display of the electrical function being generated by the function generator. The exemplary oscilloscope has a frequency range of up to 60 MHz. The oscilloscope is used as a diagnostic tool for monitoring application of the ultrasonic energy.

The exemplary amplifier 206 increases the intensity of the signal generated by the generator and has a power output of up to 20 Watts. Any standard RF amplifier can be used. The matching network 208 modifies the impedance of the incoming signal to match the impedance of the transducer 210. The matching network must be configured to the unique characteristics of the transducer 210. The exemplary transducer 210 contains a piezoelectric crystal and converts the matched, amplified electrical signaling into ultrasonic waves 212. Transducers can emit a frequency range of 20 KHz to 20 MHz. The ultrasonic waves 212 can be generated in a continuous mode or can be pulsed. A particular mode may be more desirable based on the particular application. The exemplary transducer 210 can deliver between 0.10 and 2.0 Watts of acoustic power.

Embodiments of the present invention can use different configurations of the transducer tip 142. The shape and surface area of the transducer tip 142 can be modified based on the particular application. Exemplary transducer tips for transcleral applications have circular cross-sectional areas and can have diameters ranging between 5 mm and 15 mm. In other embodiments, the transducer tip has a quasi-heart shape, hemi-spherical, or otherwise concave surface with a curvature to nearly correspond with the curvature of the scleral surface of the eye. The curvature of the eye at the scleral surface is unique as compared to the corneal surface of the eye, thus the curvature of the transducer tip can be specifically adapted for transcleral delivery. A concave tip curvature that closely approximates the curvature of the eye at the sclera optimizes the surface area of the sclera that is oppositely opposed to and thus directly exposed to the tip of the transducer, which is emitting the ultrasonic waves. This opposing curvatures enhances delivery of the pharmaceutical formulation through the sclera.

Returning to FIG. 1, during sonophoresis (another term describing the process of emitting ultrasonic waves), the ultrasonic waves emitted by the transducer 132 are translated through the coupling media 134 and the pharmaceutical formulation 138 is delivered through the sclera 104, choroid 106, and retina 108 and into the vitreous 110 where it can reach the macula 114. The blood-retina barrier contains vascularity 140, which tends to transport the pharmaceutical formulation around to other parts of the eye. Embodiments of the present invention take advantage of this transport for situations where distribution of a pharmaceutical formulation throughout other tissues of the eye, such as the retinal tissue or optic nerve 112, is desired. Further, transcieral drug delivery can more directly target tissue in the posterior region of the eye, as compared to a transcorneal route.

In a transcorneal route, a pharmaceutical agent must be transported across the cornea 102, which has multiple layers, including the epithelial layer and the stroma. In addition, the agent must diffuse through the aqueous humor 120 and travel through the pupil 116 and lens 118, or through the ciliary body. Only after crossing these portions of the eye is the agent delivered into the vitreous 110 where it can reach other regions of the eye. The transcleral route used by embodiments of the present invention allows the targeted tissue to be more directly reached. Additionally, a transcorneal route cannot take advantage of the ability of the vascularity 140 in the blood-retina barrier to distribute the agent to other tissue in the eye.

A variety of classes of pharmaceutical formulations can be delivered to the eye using embodiments of the present invention. These agents are intended to provide a variety of actions such as antibiotic, anti-viral, chemotherapeutic, cellular restoration, and gene therapeutic activities; or a combination of these actions. The classes of drugs that can be delivered include, by way of example and not limitation, hydrophilic drugs, lipophilic drugs, liposomes, dendrimers, cyclodextrans, gas encapsulated particles, ultrasound contrast agents, nanoparticles, microspheres, peptides, linear and globular proteins (up to 80 kDa), linear and globular gene therapeutic drugs of varying molecular weights, adeno-associated virus gene therapy agents, and naked RNA/DNA. As discussed above, the particular pharmaceutical formulation to be delivered to the targeted tissue within the eye affects other variables. For example, the standoff distance, transducer configuration, electrical function, frequency, coupling media, coupling well or cartridge volume, formulation concentration, exposure time, and targeted tissue, such as the macula or retina, can all be configured according to the particular pharmaceutical formulation used. In this case, these exemplary agents are to be delivered to tissue in the posterior regions of the eye, because they are designed to treat conditions requiring delivery to these regions. These target conditions can differ from conditions affecting anterior segments of the eye, such as keratitis or glaucoma.

The present invention has been described in relation to particular embodiments, which are intended in all respects to illustrate rather than restrict. Alternative embodiments will become apparent to those skilled in the art that do not depart from its scope. Many alternative embodiments exist, but are not included because of the nature of this invention. A person of ordinary skill in the art may develop alternative means for implementing the aforementioned embodiments without departing from the scope of the present invention.

It will be understood that certain features and sub-combinations of utility may be employed without reference to features and sub-combinations and are contemplated within the scope of the claims. Furthermore, the steps performed need not be performed in the order described.

The invention claimed is:

1. A method for delivering one or more pharmaceutical agents to an eye through its sclera, comprising:
filling a coupling well with a coupling media and a pharmaceutical formulation;
placing an ultrasonic-wave-generating device in contact with the coupling media and within a desired standoff distance from the sclera; and
using the ultrasonic wave-generating device to transport the pharmaceutical formulation into the eye through the sclera.

2. The method of claim 1, wherein the coupling well is a cartridge that positions the ultrasonic wave-generating device at a pre-determined standoff distance, wherein the pre-determined standoff distance is the desired standoff distance.

3. The method of claim 1, wherein the ultrasonic wave-generating device includes a transducer having a tip with a concave surface.

4. The method of claim 3, wherein the curvature of the tip of the transducer closely corresponds with the curvature of the eye.

5. The method of claim 1, wherein the ultrasonic wave-generating device is used to emit pulsed ultrasonic waves to transport the pharmaceutical formulation through the sclera.

6. The method of claim 1, wherein the ultrasonic wave-generating device is used to emit continuous ultrasonic waves to transport the pharmaceutical formulation through the sclera.

7. The method of claim 2, wherein the cartridge has a pre-determined standoff distance between about 0.5 and 1.5 centimeters.

8. The method of claim 1, wherein the eye is exposed to ultrasonic waves for an exposure time of about 10 minutes or less.

9. The method of claim 1, wherein the ultrasonic wave-generating device is operated at a frequency between about 100 kHz and 1.75 MHz.

10. An ultrasonic transcleral drug delivery system, comprising:
a function generator capable of generating electrical signals from algorithms;
an amplifier capable of increasing the intensity of the electrical signals;
a matching network capable of modifying the impedance of the electrical signals;
a transducer capable of emitting ultrasonic waves from the electrical signals, wherein the transducer has a tip shaped to enhance transport of a pharmaceutical formulation through scleral tissue;
and a coupling well capable of holding a coupling media containing the pharmaceutical formulation and adapted to allow the transducer to be positioned so as to provide a desired standoff distance from the scleral tissue.

11. The system of claim 10, wherein the coupling well is a cartridge that positions the transducer at a pre-determined standoff distance, wherein the pre-determined standoff distance is the desired standoff distance.

12. The system of claim 10, further comprising a visual display capable of graphically displaying the electrical signals.

13. The system of claim 10, wherein the transducer is capable of emitting ultrasonic waves in a continuous mode, in a pulsed mode, or in a combination of continuous and pulsed waves.

14. The system of claim 10, wherein the transducer tip shaped to enhance transport of the pharmaceutical formulation has a concave-curved tip that approximates the curvature of an eye at its sclera.

15. The system of claim 11, wherein the system further comprises a transducer-specific connector that connects the cartridge to the transducer.

16. An ultrasonic pharmaceutical-transport apparatus for delivering pharmaceutical formulations through a sclera, comprising:
an ultrasonic wave source capable of generating ultrasonic waves at a plurality of frequencies;
an ultrasonic transducer tip having a concave curvature approximating the curvature of a human eye;
a pre-configured cartridge adapted for containing a volume of a coupling media and a pharmaceutical formulation to be delivered through the sclera of the human eye, wherein the pre-configured cartridge positions the transducer tip at a pre-determined standoff distance from the sclera.

17. The apparatus of claim 16, wherein the ultrasonic wave-generating source comprises a function generator capable of generating electrical signals, an amplifier, and a matching network capable of modifying the electrical signals.

18. The apparatus of claim 16, wherein the transducer tip has a cross-sectional area with a diameter of between about 5 and 15 millimeters.

19. The apparatus of claim 16, wherein the plurality of frequencies comprises frequencies within the range between 100 kHz and 1.75 MHz.

20. The apparatus of claim 16, wherein the pre-configured cartridge is capable of being adjusted to provide for a plurality of pre-determined standoff distance settings.
